SEQUENCE LISTING (3 sequences)

SEQ ID NO: 1 - protein, 505 aa, Cerrena unicolor
  misc_feature: Cerrena unicolor laccase D1
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 2 - protein, 505 aa, Cerrena unicolor
  misc_feature: Cerrena unicolor laccase D1
AIGPVADIHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI

SEQ ID NO: 3 - protein, 583 aa, Aspergillus niger
  misc_feature: Aspergillus niger glucose oxidase
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

(19) United States
(12) Patent Application Publication
(10) Pub. No.: US 2012/0064602 A1
(43) Pub. Date: Mar. 15, 2012

(54) DISH DETERGENT COMPRISING BLEACHING ENZYMES

(76) Inventors: Pieter Augustinus, Gouda (NL); Maria Petra Van Deurzen, Delfgauw (NL); Quinta Mildred Westdijk-Patist, Spijkenisse (NL)

(21) Appl. No.: 13/321,789

(22) PCT Filed: May 20, 2010
(86) PCT No.: PCT/US10/35526

§ 371 Date: Nov. 21, 2011

(30) Foreign Application Priority Data
May 21, 2009 (US) 61180228

(51) Int. Cl. C12N 9/04; C12S 9/00; D06L 3/11; C11D 7/60; B08B 3/08; C12N 9/02

(52) U.S. Cl. 435/190; 134/25.2; 435/189; 435/264; 510/218

(57) ABSTRACT

Described are compositions and methods that involve using bleaching enzymes in dish detergents. In some preferred embodiments, the bleaching enzyme comprises at least one laccase, while in some alternative preferred embodiments, the bleaching enzyme comprises at least one glucose oxidase suitable for use in dish detergents. In some additional preferred embodiments, the dish detergents are machine dish detergents.

### PRIORITY

The present application claims priority to U.S. Provisional Patent Application Ser. No. 61/180,228, filed on May 21, 2009, which is hereby incorporated by reference.

### TECHNICAL FIELD

The present invention provides compositions and methods relating to the use of bleaching enzymes in dish detergents. In some preferred embodiments, the bleaching enzyme comprises at least one laccase, while in some alternative preferred embodiments, the bleaching enzyme comprises at least one glucose oxidase. In some additional preferred embodiments, the dish detergents are machine dish detergents.

### BACKGROUND

Laccases are copper-containing enzymes produced by various organisms. These enzymes are good oxidizing agents in the presence of oxygen and find use in many applications, including pulp and textiles bleaching, treatment of pulp waste water, de-inking, industrial color removal, bleaching laundry detergents, oral care teeth whiteners, and as catalysts or facilitators for polymerization and oxidation reactions. In some applications, phenol oxidizing enzymes find use as aids in the removal of stains (e.g., food stains), from clothes during detergent washing.

Laccases are known to be produced by a wide variety of fungi, including species of the genera Aspergillus, Neurospora, Podospora, Botrytis, Pleurotus, Fornes, Phlebia, Trametes, Polyporus, Stachybotrys, Rhizoctonia, Bipolaris, Curvularia, Amerosporium, and Lentinus. Most laccases exhibit pH optima in the acidic pH range while being inactive in neutral or alkaline pHs.

For many applications, the oxidizing efficiency of a laccase can be improved through the use of a mediator, also known as an enhancing agent. Systems that include a laccase and a mediator are known in the art as laccase-mediator systems (LMS). The same compounds can also be used to activate or initiate the action of laccase.

There are several known mediators for use in laccase-mediator systems. These include 1-hydroxybenzotriazole (HBT), 2,2′-azinobis (3-ethylbenzothiazoline-6-sulfinic acid) (ABTS), N-hydroxyacetanilide (NHA), N-acetyl-N-phenylhydroxylamine (NEIAA), 3-hydroxy 1,2,3-benzotriazin-4(3H)-one (HBTO), and violuric acid (VIO). In addition, there are several compounds containing NH—OH or N—O that have been found to be useful as mediators. Functional groups and substituents have large effects on mediator efficiency. Even within the same class of compounds, a substituent can change the laccase specificity towards a substrate, thereby greatly increasing or decreasing mediator efficiency. In addition, some mediators are effective for one particular application but are unsuitable for another application.

### SUMMARY

The present invention provides compositions and methods for using bleaching enzymes in dish detergents. In some embodiments, the bleaching enzyme is at least one laccase, while in other embodiments, the bleaching enzyme is at least one glucose oxidase. In further embodiments, the bleaching enzyme is at least one laccase and at least one glucose oxidase. In some embodiments, the dish detergents are machine (i.e., automatic) dish detergents. In further embodiments, the dish detergents provide good bleaching performance over a range of pH values. In some embodiments, the pH is about 7, while in other embodiments, the pH is about 10.

In one aspect, a cleaning composition is provided, comprising a bleaching enzyme selected from a laccase and a glucose oxidase, wherein the bleaching enzyme is capable of bleaching a stain on a dishware item in the absence of a chemical bleaching agent.

In some embodiments, the enzyme is a laccase. In some embodiments, the laccase is a Cerrena unicolor laccase. In some embodiments, the laccase is Cerrena unicolor laccase D1.

In particular embodiments, the laccase has at least 90% amino acid sequence identity to the amino acid sequence of SEQ ID NO: 1 or SEQ ID NO: 2. In more particular embodiments, the laccase has the amino acid sequence of SEQ ID NO: 1 or SEQ ID NO: 2.

In some embodiments, the enzyme is a laccase and the cleaning composition further comprises the mediator syringonitrile.

In some embodiments, the enzyme is a glucose oxidase. In some embodiments, the glucose oxidase is an Aspergillus niger glucose oxidase

In particular embodiments, the glucose oxidase has at least 90% amino acid sequence identity to the amino acid sequence of SEQ ID NO: 3. In more particular embodiments, the glucose oxidase has the amino acid sequence of SEQ ID NO: 3.

In another aspect, a method for cleaning a stain from the surface of an object is provided, comprising: providing a cleaning composition comprising a bleaching enzyme selected from a laccase and a glucose oxidase, and contacting the object with the cleaning composition, wherein the contacting removes at least a portion of the stain from the surface of the object.

In some embodiments, the object is a dishware item. In some embodiments, the method is performed in an automatic dishwasher.

In some embodiments, the method is performed at neutral pH. In some embodiments, the method is performed at pH or 7 or more.

In some embodiments, the enzyme is a laccase. In some embodiments, the laccase is a Cerrena unicolor laccase.

In particular embodiments, the laccase has at least 90% amino acid sequence identity to the amino acid sequence of SEQ ID NO: 1 or SEQ ID NO: 2. In still more particular embodiments, the laccase has the amino acid sequence of SEQ ID NO: 1 or SEQ ID NO: 2.

In some embodiments, the enzyme is a laccase and the cleaning composition comprises the mediator syringonitrile.

In some embodiments, the enzyme is a glucose oxidase. In some embodiments, the glucose oxidase is an Aspergillus niger glucose oxidase.

In particular embodiments, the glucose oxidase has at least 90% amino acid sequence identity to the amino acid sequence of SEQ ID NO: 3. In still more particular embodiments, the glucose oxidase has the amino acid sequence of SEQ ID NO: 3.

These and other aspects and embodiments of the present compositions and method will be apparent from the present description.

### DESCRIPTION

The present invention provides compositions and methods for use of bleaching enzymes in dish detergents. In some embodiments, the bleaching enzyme comprises at least one laccase, while in alternative embodiments, the bleaching enzyme comprises at least one glucose oxidase suitable for use in dish detergents. In additional embodiments, the dish detergents are automatic (i.e., machine) dish detergents.

In some embodiments, the compositions and methods are for use in neutral pH conditions, i.e., a pH of from about 6 to 9, from about 6 to 8.5, from about 6 to 8, or even from about 6.5 to about 7.5. In contrast, conventional compositions and methods involve more alkaline pH conditions, e.g., a pH of 10 or more, a pH of 9.2 or more, or even a pH of 8.5 or more. The use of more neutral pH conditions offers certain benefits, e.g., in terms of shine.

### Definitions

Unless defined otherwise herein, all technical and scientific terms used herein have the same meaning as commonly understood by one of ordinary skill in the art to which this invention belongs. Although any methods and materials similar or equivalent to those described herein find use in the practice of the present invention, the preferred methods and materials are described herein. Also, as used herein, the singular terms “a,” “an,” and “the” include the plural reference unless the context clearly indicates otherwise. Unless otherwise indicated, nucleic acids are written left-to-right in 5′ to 3′ orientation; amino acid sequences are written left-to-right in amino to carboxy orientation. It is to be understood that this invention is not limited to the particular methodology, protocols, and reagents described, as these may vary, depending upon the context they are used by those of skill in the art. The headings provided herein are not limitations of the various aspects or embodiments of the invention which can be had by reference to the specification as a whole.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All publications cited herein are expressly incorporated herein by reference for the purpose of describing and disclosing compositions and methodologies which might be used in connection with the invention.

The terms defined immediately below are more fully defined by reference to the specification as a whole:

As used herein, “cleaning compositions” and “cleaning formulations” refer to compositions that find use in the removal of undesired materials or compounds from items to be cleaned, such as fabrics, dishes, utensils, hard surfaces, anf the like. The terms encompass any type and form of cleaning composition (e.g., liquid, gel, granule, or spray composition), so long as the components in the composition are compatible with the laccase, glucose oxidase, other bleaching enzyme(s), or other additional enzymes, used in the composition. The selection of a particular cleaning composition is readily made by considering the type of surface or the article to be cleaned, and the desired form of the composition for the cleaning application.

The terms further refer to any composition that is suited for cleaning and/or bleaching any object and/or surface. It is intended that the terms include, but are not limited to detergent compositions for dish detergents. In some particularly preferred embodiments, the dish detergents are automatic (i.e., machine) dish detergents, e.g., for automatic dish washing (ADW).

Indeed, the term “cleaning composition” as used herein, includes (unless otherwise indicated), granular or powder-form all-purpose or heavy-duty washing agents, especially cleaning detergents; liquid, gel or paste-form all-purpose washing agents, including hand dishwashing agents or light duty dishwashing agents, especially those of the high-foaming type; machine dishwashing agents, including the various tablet, granular, liquid and rinse-aid types for household and institutional use; liquid cleaning and disinfecting agents, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the terms “detergent composition” and “detergent formulation” are used with reference to admixtures that are intended for use in a wash medium for the cleaning of soiled objects. In some embodiments, the term refers to detergents, such as those used to clean dishes, cutlery, etc. (e.g., “dishwashing detergents”). It is not intended that the presently contemplated compositions be limited to any particular detergent formulation or composition. Indeed, it is intended that in addition to laccase, glucose oxidase, and/or other bleaching enzyme-containing compositions, the term encompasses detergents that contain, e.g., surfactants, transferase(s), hydrolytic enzymes, builders, bleaching agents, bleach activators, bluing agents and fluorescent dyes, caking inhibitors, masking agents, enzyme activators, antioxidants, and/or solubilizers.

As used herein, the term “dishwashing composition” refers to all forms of compositions for cleaning dishes, including but not limited to granular and liquid forms. Indeed, as used herein, “dishwashing composition” refers to all forms of compositions for cleaning dishware, including cutlery, including but not limited to granular and liquid forms. It is not intended that the present invention be limited to any particular type or dishware composition. Indeed, the present invention finds use in cleaning dishware (e.g., dishes, including, but not limited to plates, cups, glasses, bowls, etc.) and cutlery (e.g., utensils, including but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, the term “hard surface cleaning composition,” refers to detergent compositions for cleaning hard surfaces such as floors, walls, tile, stainless steel vessels (e.g., fermentation tanks), bath and kitchen fixtures, and the like. Such compositions are provided in any form, including but not limited to solids, liquids, emulsions, etc.

As used herein, the term “bleaching” refers to the treatment of a material, item (e.g., fabric, laundry, pulp, dishes, etc.) or surface for a sufficient length of time and under appropriate pH and temperature conditions to effect a brightening (i.e., whitening) and/or cleaning of the material. Examples of chemicals suitable for bleaching that find use in various compositions of the present invention include but are not limited to ClO₂, H₂O₂, peracids, NO₂, etc.

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

As used herein, the term “compatible,” means that the cleaning composition materials do not reduce the enzymatic activity of the protease enzyme(s) provided herein to such an extent that the protease(s) is/are not effective as desired during normal use situations. Specific cleaning composition materials are exemplified in detail hereinafter.

As used herein, “non-phosphate containing dishwashing detergents” are detergents that contain no more than 0.5% phosphorus (i.e., phosphorus is a trace element).

As used herein, “neutral pH compositions” and “neutral pH detergents” encompass detergents that are effective at a pH in the neutral range. i.e., a pH of from about 6 to 9, from about 6 to 8.5, from about 6 to 8, or even from about 6.5 to about 7.5.

As used herein, the “wash performance” of a mutant (or variant) enzyme refers to the contribution of an enzyme to dishwashing that provides additional cleaning performance to the detergent without the addition of the enzyme to the composition. Wash performance is determined under relevant washing conditions.

As used herein, the term “relevant washing conditions” refers to the conditions, particularly washing temperature, time, washing mechanics, sud concentration, type of detergent and water hardness, that are actually used in households in a dish detergent market segment.

As used herein, the term “improved wash performance” is used to indicate that a better end result is obtained in stain removal from dishware and/or cutlery under relevant washing conditions, or that less enzyme, on weight basis, is needed to obtain the same end result relative to results obtained in the absence of the enzyme.

Wash performance of enzymes is conveniently measured by their ability to remove certain representative stains (containing materials or compounds) under appropriate test conditions. In these test systems, other relevant factors, such as detergent composition, sud concentration, water hardness, washing mechanics, time, pH, and/or temperature, can be controlled in such a way that conditions typical for household application in a certain market segment are imitated. The laboratory application test system described herein is representative for household applications. Thus, the methods provided herein facilitate the testing of large amounts of different enzymes and the selection of those enzymes which are particularly suitable for a specific type of detergent application. In this way “tailor made” enzymes for specific application conditions are easily selected.

As used herein, “effective amount of enzyme” refers to the quantity of enzyme necessary to achieve the enzymatic activity required in the specific application. Such effective amounts are readily ascertained by one of ordinary skill in the art and are based on many factors, such as the particular enzyme used, the cleaning application, the specific composition of the cleaning composition, and whether a liquid or dry (e.g., granular) composition is required, and the like.

As used herein, the phrase “detergent stability” refers to the stability of components, including enzymes, in a detergent composition. In some embodiments, the stability is assessed during the use of the detergent, while in other embodiments, the term refers to the stability of a detergent composition during storage (and/or shipment).

As used herein, the expression “in the absence of a chemical bleaching agent” means in the absence of a chemical/small molecule bleaching agent, such as hypochlorite, hydrogen peroxide (or a compound capable of generating hydrogen peroxide, such as perborate, percarbonate, persulfate, pyrophosphate, and urea peroxide), N,N,N′N′-tetraacetylethylenediamine, bromate, nonanoyloxybenzenesulfonate, and the like.

As used herein, “chemical” bleaching agents are small molecules, which are to be distinguished from macromolecular bleaching agents, such as enzymes.

As used herein, the term “specific performance” refers to the ability of a subject composition (or enzyme, therein) to clean particular stains, as a function of unit of active protein. In some preferred embodiments, the specific performance is determined using stains such as egg yolk, egg/milk, minced meat, tea, milk, porridge, tea, tomato, coffee, etc.

As used herein, the terms “purified” and “isolated” refer to the removal of contaminants from a sample. For example, an enzyme of interest is purified by removal of contaminating proteins and other compounds within a solution or preparation that are not the enzyme of interest. In some embodiments, recombinant enzymes of interest are expressed in bacterial or fungal host cells and these recombinant enzymes of interest are purified by the removal of other host cell constituents; the percent of recombinant enzyme of interest polypeptides is thereby increased in the sample.

As used herein, the term “protein of interest,” refers to a protein (e.g., an enzyme or “enzyme of interest”) which is being analyzed, identified and/or modified. Naturally-occurring, as well as recombinant (e.g., mutant, variant) proteins find use in the present invention.

As used herein, the term “protein” refers to any composition comprised of amino acids and recognized as a protein by those of skill in the art. The terms “protein,” “peptide” and “polypeptide” are used interchangeably herein. Wherein a peptide is a portion of a protein, those skilled in the art understand the use of the term in context.

As used herein, “functionally and/or structurally similar proteins” are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms (e.g., a bacterial enzyme and a fungal enzyme). In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the enzymes of the present invention). In further embodiments, the term encompasses proteins that are immunologically cross-reactive.

### Glucose Oxidase

As used herein, the term “glucose oxidase”refers to any enzyme encompassed by enzyme classification (EC) 1.1.3.4. Glucose oxidase enzymes catalyze the oxidation of beta-D-glucose into D-glucono-1,5-lactone, which is then hydrolyzed to glucanonic acid. Any suitable glucose oxidase finds use in the present invention. It is not intended that the present invention be limited to any particular glucose oxidase, nor glucose oxidase produced by any particular organism. In addition, naturally-occurring, as well as recombinantly-produced glucose oxidases find use in the present invention. It is also intended that any suitable commercially available glucose oxidase will find use in the present invention. In some preferred embodiments, the glucose oxidase of the present invention has the amino acid sequence of SEQ ID NO: 3. In other preferred embodiments, the glucose oxidase has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% identical to the amino acid sequence of SEQ ID NO: 3.

### Laccases

As used herein, the terms “laccases” and “laccase-related enzymes” include any laccase enzyme encompassed by EC 1.10.3.2. The laccase enzymes are typically obtained from microorganisms or plants. In some embodiments, the microbial laccase is obtained from bacteria or fungi (including filamentous fungi and yeasts). Suitable sources include, but are not limited to strains of Aspergillus, Neurospora (e.g., N. crassa), Podospora, Botrytis, Collybia, Cerrena, Stachybotrys, Panus (e.g., Panus rudis), Theilava, Fomes, Lentinus, Pleurotus, Trametes (e.g., T. villosa and T. versicolor), Rhizoctonia (e.g., R. solani), Coprinus (e.g., C. plicatilis and C. cinereus), Psatyrella, Myceliophthora (e.g., M. thermonhila), Schytalidium, Phlebia (e.g., P. radita; see, e.g., WO 92/01046), Coriolus (e.g., C. hirsutus; see, e.g., JP 2-238885), Spongipellis, Polyporus, Ceriporiopsis subvermispora, Ganoderma tsunodae, and Trichoderma. Indeed it is not intended that the present invention be limited to a particular laccase obtained from any particular source, as any suitable laccase finds use in the present invention. In addition, naturally-occurring and/or recombinantly-produced laccases find use in the present invention. Thus, it is not intended that the laccase be limited to any particular method of production. In some preferred embodiments, the laccases of the present invention include but are not limited to the laccases described in U.S. Patent Pub. No. 2008189871, U.S. Patent Pub. No. 2008196173, or International Patent Pub. No. WO 2008/076322, herein incorporated by reference in its entirety. In some preferred embodiments, the laccase has the amino acid sequence SEQ ID NO: 1 or 2. In other preferred embodiments, the laccase has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% identical to the amino acid sequence of SEQ ID NOs: 1 or 2.

As indicated above, industrial applications of laccases include bleaching of pulp and paper and textile bleaching (e.g., indigo-dyed denim fabrics), hair dyeing (see, e.g., WO 95/33836 and WO 95/33837), wool dyeing (EP 0 504 005), bleaching of textiles, fibers, yarns and the like, treatment of waste water, the delignification of pulp, depolymerization of high molecular weight aggregates, deinking waste paper, polymerization of aromatic compounds, radical mediated polymerization and cross-linking reactions (e.g., paints, coatings, biomaterials), the activation of dyes and to couple organic compounds, as well as in cleaning compositions. In some particularly preferred embodiments, the present invention provides dish washing detergents comprising laccases.

As described herein, the laccases are capable of oxidizing a wide variety of colored compounds having different chemical structures, using oxygen as the electron acceptor. Accordingly, the laccases presented herein can be used in applications where it is desirable to modify the color associated with colored compounds, such as in cleaning (e.g., for removing the food stains). In some embodiments, a mediator or enhancer is used to obtain desirable effects. In some embodiments, the laccase mediators are used as sanitization and antimicrobial agents. The mediators may be used independently of the enzymes or in conjunction with the enzymes.

### Mediators

In some embodiments, the enzymatic oxidation system further comprises one or more chemical mediator agents which enhance the activity of the laccase enzyme. The term “chemical mediator” (or “mediator”) is defined herein as a chemical compound which acts as a redox mediator to effectively shuttle electrons between the enzyme exhibiting oxidase activity and the dye. Chemical mediators are also known in the art as “enhancers” and “accelerators.”

In some embodiments, the chemical mediator is a phenolic compound (e.g., methyl syringate, and related compounds, as described in WO 95/01426 and WO 96/12845). In some other embodiments, the chemical mediator is an N-hydroxy compound, an N-oxime compound, or an N-oxide compound, for example, N-hydroxybenzotriazole, violuric acid, or N-hydroxyacetanilide. In some alternative embodiments, the chemical mediator is a phenoxazine/phenothiazine compound (e.g., phenothiazine-10-propionate). In some further embodiments, the chemical mediator is 2,2′-azinobis-(3-ethylbenzothiazoline-6-sulfonic acid) (ABTS). In still some additional embodiments, the mediator is acetosyringone, methyl syringate, ethyl syringate, propyl syringate, butyl syringate, hexyl syringate, or octyl syringate. Indeed, it is not intended that the present invention be limited a particular mediator, as other suitable chemical mediators are well known in the art (see, e.g., WO 95/01426). In some embodiments, the mediator is 4-cyano-2,6-dimethoxyphenol, 4-carboxamido-2,6-dimethoxyphenol or an N-substituted derivative thereof such as, for example, 4-(N-methyl carboxamido)-2,6-dimethoxyphenol, 4-[N-(2-hydroxyethyl) carboxamido]-2,6-dimethoxyphenol, or 4-(N,N-dimethyl carboxamido)-2,6-dimethoxyphenol.

In some embodiments, the mediator used in the present invention may be described by the following formula:

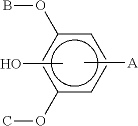

in which formula A is a group such as —R, -D, —CH═CH-D, —CH═CH—CH═CH-D, —CH═N-D, —N═N-D, or —N═CH-D, in which D is selected from the group consisting of —CO-E, —SO₂-E, —CN, —NXY, and —N⁺XYZ, in which E may be —H, —OH, —R, —OR, or —NXY, and X and Y and Z may be identical or different and selected from —H, —OH, —OR and —R; R being a C₁-C₁₆ alkyl, preferably a C₁-C₈ alkyl, which alkyl may be saturated or unsaturated, branched or unbranched and optionally substituted with a carboxy, sulfo or amino group; and B and C may be the same or different and selected from Cₘ H₂ₘ₊₁; 1≦m≦5.

In some embodiments, A in the above mentioned formula is —CN or —CO-E, in which E may be —H, —OH, —R, —OR, or —NXY, where X and Y are identical or different and selected from —H, —OH, —OR and —R, R being a C₁-C₁₆ alkyl, preferably a C₁-C₈ alkyl, which alkyl is saturated or unsaturated, branched or unbranched and optionally substituted with a carboxy, sulfo or amino group; and B and C are the same or different and selected from Cₘ H₂ₘ₊₁; 1≦m≦5. In some alternative embodiments, formula A is placed meta to the hydroxy group instead of being placed in the para-position as shown.

In some embodiments, the mediator is acetosyringone, methylsyringate, ethylsyringate, propylsyringate, butylsyringate, hexylsyringate, or octylsyringate. In some alternative embodiments, the mediator is 4-cyano-2,6-dimethoxyphenol, 4-carboxamido-2,6-dimethoxyphenol or a N-substituted derivative thereof such as 4-(N-methyl carboxamido)-2,6-dimethoxyphenol, 4-[N-(2-hydroxyethyl) carboxamido]-2,6-dimethoxyphenol, or 4-(N,N-dimethyl carboxamido)-2,6-dimethoxyphenol. The mediators of the present invention are prepared using any suitable method known in the art (see, e.g., WO 97/11217, WO 96/12845, and U.S. Pat. No. 5,752,980). In addition, the chosen mediators are used in any suitable concentration, as determined by the user.

### Experimental

The following Example is provided in order to demonstrate and further illustrate certain preferred embodiments and aspects of the present invention and should not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); by (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); a.p. or ap (active protein); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N′N′-tetraacetylethylenediamine); w/v (weight to volume); v/v (volume to volume); MS (mass spectroscopy); SR (soil or stain removal); GH (German Hardness); STPP (tri-polyphosphate); MGDA (methylglycinediacetic acid); TNC (tri-sodium citrate); AATCC (American Association of Textile and Coloring Chemists); WFK (wfk Testgewebe GmbH, Bruggen-Bracht, Germany); Center for Test Materials (Center for Test Materials, Vlaardingen, the Netherlands); Amersham (Amersham Life Science, Inc. Arlington Heights, Ill.); Sigma (Sigma Chemical Co., St. Louis, Mo.); Sorvall (Sorvall Instruments, a subsidiary of DuPont Co., Biotechnology Systems, Wilmington, Del.); Roche (Hoffmann La Roche, Inc., Nutley, N.J.); Minolta (Konica Minolta, Ramsey, N.J.); Zeiss (Carl Zeiss, Inc., Thornwood, N.Y.); Henkel (Henkel, GmbH, Dusseldorf, Germany); Genencor (Danisco US Inc., Genencor Division, Palo Alto, Calif.); and Reckitt Benckiser, Berks, United Kingdom).

### EXAMPLE 1

### Dishwashing Performance of Laccase and Glucose Oxidase

The performance of a Cerrena unicolor laccase and an Aspergillus niger glucose oxidase was tested under various automatic dishwashing conditions.

The amino acid sequence of the mature Cerrena unicolor laccase is show, below, as SEQ ID NO: 1. This sequence is similar to the amino acid sequence of laccase D1 from strain CBS154.29 (SEQ ID NO: 2) that is described in, e.g., US2008189871, US2008196173, and WO2008076322, which references are hereby incorporated by reference in their entirety. The single difference between these sequences (Leu or Ile at position 8) is underlined. This difference is not expected to affect protein structure or function, and both laccase sequences are herein referred to as Cerrena unicolor laccase D1. The C-terminal residues shown in italics are encoded as part of the laccase polypeptide but may not be present as part of the active polypeptide, e.g., due to cleavage or other processing.

(SEQ ID NO: 1)
AIGPVADLHIVNKDLAPDGVQRPTVLAGGTFPGILITGQKGDNFQLNVID
DLTDDRMLTPTSIHWHGFFQKGTAWADGPAFVTQCPIIADNSFLYDFDVP
DQAGTFWYHSHLSTQYCDGLRGAFVVYDPNDPHKDLYDVDDGGTVITLAD
WYHVLAQTVVGAATPDSTLINGLGRSQTGPADAELAVISVEHNKRYRFRL
VSISCDPNFTFSVDGHNMTVIEVDGVNTRPLTVDSIQIFAGQRYSFVLNA
NQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSV
PALLQILSGAQNAQDLLPTGSYIGLELGKVVELVIPPLAVGGPHPFHLHG
HNFWVVRSAGSDEYNFDDAILRDVVSIGAGTDEVTIRFVTDNPGPWFLHC
HIDWHLEAGLAIVFAEGINQTAAANPTPQAWDELCPKYNGLSASQKVKPK
KGTAI
(SEQ ID NO: 2)
AIGPVADIHIVNKDLAPDGVQRPTVLAGGTFPGTLITGQKGDNFQLNVID
DLTDDRMLTPTSIHWHGFFQKGTAWADGPAFVTQCPIIADNSFLYDFDVP
DQAGTFWYHSHLSTQYCDGLRGAFVVYDPNDPHKDLYDVDDGGTVITLAD
WYHVLAQTVVGAATPDSTLINGLGRSQTGPADAELAVISVEHNKRYRFRL
VSISCDPNFTFSVDGHNMTVIEVDGVNTRPLTVDSIQIFAGQRYSFVLNA
NQPEDNYWIRAMPNIGRNTTTLDGKNAAILRYKNASVEEPKTVGGPAQSP
LNEADLRPLVPAPVPGNAVPGGADINHRLNLIFSNGLFSINNASFTNPSV
PALLQILSGAQNAQDLLPTGSYIGLELGKVVELVIPPLAVGGPHPFHLHG
HNFWVVRSAGSDEYNFDDAILRDVVSIGAGTDEVTIRFVTDNPGPWFLHC
HIDWHLEAGLAIVFAEGINQTAAANPTPQAWDELCPKYNGLSASQKVKPK
KGTAI

The amino acid sequence of the mature glucose oxidase (i.e., MULTIFECT™ GO 5000L, Danisco US Inc., Palo Alto, Calif., USA) is show, below, as SEQ ID NO: 3.

(SEQ ID NO: 3)
SNGIEASLLTDPKDVSGRTVDYIIAGGGLTGLTTAARLTENPNISVLVIE
SGSYESDRGPIIEDLNAYGDIFGSSVDHAYETVELATNNQTALIRSGNGL
GGSTLVNGGTWTRPHKAQVDSWETVFGNEGWNWDNVAAYSLQAERARAPN
AKQIAAGHYFNASCHGVNGTVHAGPRDTGDDYSPIVKALMSAVEDRGVPI
KKDFGCGDPHGVSMFPNTLHEDQVRSDAAREWLLPNYQRPNLQVLIGQYV
GKVLLSQNGTTPRAVGVEFGTHKGNTHNVYAKHEVLLAAGSAVSPTILEY
SGIGMKSILEPLGIDTVVDLPVGLNLQDQTTATVRSRITSAGAGQGQAAW
FATFNETFGDYSEKAHELLNTKLEQWAEEAVARGGFHNTTALLIQYENYR
DWIVNHNVAYSELFLDTAGVASFDVWDLLPFTRGYVHILDKDPYLHHFAY
DPQYFLNELDLLGQAAATQLARNISNSGAMQTYFAGETIPGDNLAYDADL
SAWTEYIPYHFRPNYHGVGTCSMMPKEMGGVVDNAARVYGVQGLRVIDGS
IPPTQMSSHVMTVFYAMALKISDAILEDYASMQ

The base dish detergent used was commercially available GSM-B detergent, adjusted to pH 7 (IEC 60436 Dishwasher Reference Detergent Type B without TAED and without perborate monohydrate (wfk Testmaterials). The stain type used was DM-11 Tea on Melamine Tiles (Standard soiled Tea tile, for measuring bleach performance of dishwash detergents (Center for Test Materials).

The washing tests were performed using a Dish-O-Meter assay, substantially as described in WO 2008/010925. Three Tea tiles DM-11 were added per beaker. A defined amount (20 g/7 L) of the detergent was used. The temperature tested was 50° C., ramping from room temperature to 50° C. in 15 minutes, followed by 30 minutes at 50° C. The water hardness was 21° GH. After washing, rinsing was done in cold tap water for 3 minutes and the tiles were allowed to dry. Stain removal was measured using a Tristimulus Minolta Meter CR-400.

% Soil Removal was calculated as (ΔE value_{after washing-before washing})/(ΔE_{ref-before washing})×100% , where ΔE=√(L−L_{ref})²+(a−a_{ref})²+(b−b_{ref})²L_{ref}=96.0, a_{ref}=0.55, b_{ref}=1.95

The following treatment conditions were evaluated: GSM-B detergent, no bleach, pH 7; GSM-B+2% TAED+14% Percarbonate, pH 7; GSM-B+2% TAED+14% Percarbonate, pH 10; GSM-B+0.07 g/l Syringonitrile+0.15 g/1 Laccase pH 7; GSM-B+0.14 g/l Syringonitrile+0.30 g/l Laccase pH 7; GSM-B+0.35 g/l Syringonitrile+0.75 g/l Laccase pH 7; GSM-B+0.3 g/l Glucose+0.2 g/l TAED+2 ppm Glucose oxidase, pH 7; GSM-B+0.3 g/l Glucose+0.2 g/l TAED+5 ppm Glucose oxidase, pH 7; and GSM-B+0.3 g/l Glucose+0.2 g/l TAED+10 ppm Glucose oxidase, pH 7. The experiments were set up as follows:

GSM-B, no bleach pH 7: Preparation of solutions: Detergent solution: 10.0 gram GSM-B and 4.13 gram citric acid was added to 5 liters water of 21° GH. 250 mL of detergent solution was added to the disherometer beaker. The reaction was started by adding the tea tiles DM-11 to the beaker and starting the disherometer program.

GSM-B+2% TAED+14% Percarbonate, pH 7: Preparation of solutions: Detergent solution: 8.0 gram GSM-B+0.19 gram TAED+1.34 gram Percarbonate+4.0 gram citric acid were added to 5 liters water of 21° GH. 250 mL of detergent solution was added to the disherometer beaker. The reaction was started by adding the tea tiles DM-11 to the beaker and starting the disherometer program.

GSM-B+2% TAED+14% Percarbonate, pH 10: Preparation of solutions: Detergent solution:10.0 gram GSM-B1+1.67 gram Percarbonate+0.24 gram TAED+2.47 gram citric acid were added to 5 liters water of 21 ° GH. 250 mL of detergent solution was added to the disherometer beaker. The reaction was started by adding the tea tiles DM-11 to the beaker and starting the disherometer program.

GSM-B+Syringonitrile 0.07 g/l+Laccasse 0.15 g/l: Preparation of solutions: Detergent solution: 10.0 gram GSM-B and 4.13 gram citric acid were added to 5 liters water of 21° GH. 250 mL of detergent solution was added to the disherometer beaker. Subsequently 0.018 g syringonitrile and 0.038 g Laccase were added to the beaker. The reaction was started by adding the tea tiles DM-11 to the beaker and starting the disherometer program.

GSM-B+Syringonitrile 0.14 g/l+Laccasse 0.30 g/l: Preparation of solutions: Detergent solution: 10.0 gram GSM-B1 and 4.13 gram citric acid were added to 5 liters water of 21 ° GH. 250 mL of detergent solution was added to the disherometer beaker. Subsequently.0.035 g syringonitrile and 0.075 g Laccase were added to the beaker. The reaction was started by adding the tea tiles DM-11 to the beaker and starting the disherometer program.

GSM-B+Syringonitrile 0.35 g/l+Laccasse 0.75 g/l: Preparation of solutions: Detergent solution: 10.0 gram GSM-B1 and 4.13 gram citric acid were added to 5 liters water of 21° GH. 250 mL of detergent solution was added to the disherometer beaker. Subsequently 0.088 g syringonitrile and 0.188 g Laccase were added to the beaker. The reaction was started by adding the tea tiles DM-11 to the beaker and starting the disherometer program.

GSM-B+0.3 g/l Glucose+0.2 g/l TAED+2 ppm Glucose oxidase, pH 7: Preparation of solutions: Detergent solution: 10.0 gram GSM-B1+4.13 gram citric acid were added to 5 liters water of 21° GH. Enzyme stock solution: 8.43 gram Glucose oxidase was added to 100 ml tap water. 250 mL of detergent solution was added to the disherometer beaker. Subsequently, 0.075 g of glucose, 0.05 g of TAED and 0.213 mL of enzyme stock solution were added. The reaction was started by adding the tea tiles DM-11 to the beaker and starting the disherometer program.

GSM-B+0.3 g/l Glucose+0.2 g/l TAED+5 ppm Glucose oxidase, pH 7: Preparation of solutions: Detergent solution: 10.0 gram GSM-B+4.13 gram citric acid was added to 5 liters water of 21° GH. Enzyme stock solution: 8.43 gram Glucose oxidase was added to 100 ml tap water. 250 mL of detergent solution was added to the disherometer beaker. Subsequently, 0.075 g of glucose, 0.05 g of TAED and 0.53 mL of enzyme stock solution were added. The reaction was started by adding the tea tiles DM-11 to the beaker and starting the disherometer program.

GSM-B+0.3 g/l Glucose+0.2 g/l TAED+10 ppm Glucose oxidase, pH 7: Preparation of solutions: Detergent solution: 10.0 gram GSM-B+4.13 gram citric acid was added to 5 liters water of 21° GH. Enzyme stock solution: 8.43 gram Glucose oxidase was added to 100 ml tap water. 250 mL of detergent solution was added to the disherometer beaker. Subsequently, 0.075 g of glucose, 0.05 g of TAED and 1.06 mL of enzyme stock solution were added. The reaction was started by adding the tea tiles DM-11 to the beaker and starting the disherometer program.

The results are shown in Table 1. The used of laccase or glucose oxidase enzymes (an appropriate mediators/substrates) clearly improved soil removal compared to the control treatment.

TABLE 1
Soil Removal Performance of Laccase and Glucose Oxidase at Neutral pH

| | % Soil | |
|---|---|---|
| | Removal | Standard |
| Treatment | (n = 9) | Deviation |
| GSM B, no bleach, pH 7 | 13.2 | 1.4 |
| GSM B + 2% TAED + 14% Percarbonate, pH 7 | 32.9 | 0.7 |
| GSM B + 2% TAED + 14% Percarbonate, pH 10 | 43.6 | 2.0 |
| GSM B + Syringonitrile 0.07 g/l + Laccasse 0.15 g/l, pH 7 | 19.6 | 0.5 |
| GSM B + Syringonitrile 0.14 g/l + Laccasse 0.30 g/l, pH 7 | 23.6 | 0.9 |
| GSM B + Syringonitrile 0.35 g/l + Laccasse 0.75 g/l, pH 7 | 25.1 | 1.3 |
| GSM B + Glucose 0.3 g/l + TAED 0.2 g/1 + 2 ppm Glucose oxidase, pH 7 | 19.3 | 1.8 |
| GSM B + Glucose 0.3 g/l + TAED 0.2 g/1 + 5 ppm Glucose oxidase, pH 7 | 21.5 | 1.6 |
| GSM B + Glucose 0.3 g/l + TAED 0.2 g/1 + 10 ppm Glucose oxidase, pH 7 | 23.4 | 1.3 |

1. A cleaning composition comprising a bleaching enzyme selected from a laccase and a glucose oxidase, wherein the bleaching enzyme is capable of bleaching a stain on a dishware item in the absence of a chemical bleaching agent.

2. The composition of claim 1, wherein the enzyme is a laccase.

3. The composition of claim 2, wherein the laccase is a Cerrena unicolor laccase

4. The composition of claim 3, wherein the laccase is Cerrena unicolor laccase D1.

5. The composition of claim 2, wherein the laccase has at least 90% amino acid sequence identity to the amino acid sequence of SEQ ID NO: 1 or SEQ ID NO: 2.

6. The composition of claim 5, wherein the laccase has the amino acid sequence of SEQ ID NO: 1 or SEQ ID NO: 2.

7. The composition of claim 2, further comprising the mediator syringonitrile.

8. The composition of claim 1, wherein the enzyme is a glucose oxidase.

9. The composition of claim 8, wherein the glucose oxidase is an Aspergillus niger glucose oxidase

10. The composition of claim 8, wherein the glucose oxidase has at least 90% amino acid sequence identity to the amino acid sequence of SEQ ID NO: 3.

11. The composition of claim 10, wherein the glucose oxidase has the amino acid sequence of SEQ ID NO: 3.

12. A method for cleaning a stain from the surface of an object comprising:
providing a cleaning composition comprising a bleaching enzyme selected from a laccase and a glucose oxidase, and
contacting the object with the cleaning composition,
wherein the contacting removes at least a portion of the stain from the surface of the object.

13. The method of claim 12, wherein the object is a dishware item.

14. The method of claim 12, wherein the method is performed in an automatic dishwasher.

15. The method of claim 12, wherein the method is performed at neutral pH.

16. The method of claim 12, wherein the method is performed at pH or 7 or more.

17. The method of claim 12, wherein the enzyme is a laccase.

18. The method of claim 17, wherein the laccase is a Cerrena unicolor laccase.

19. The method of claim 17, wherein the laccase has at least 90% amino acid sequence identity to the amino acid sequence of SEQ ID NO: 1 or SEQ ID NO: 2.

20. The method of claim 19, wherein the laccase has the amino acid sequence of SEQ ID NO: 1 or SEQ ID NO: 2.

21. The method of claim 12, wherein the enzyme is a laccase and the cleaning composition comprises the mediator syringonitrile.

22. The method of claim 12, wherein the enzyme is a glucose oxidase.

23. The method of claim 22, wherein the glucose oxidase is an Aspergillus niger glucose oxidase.

24. The method of claim 22, wherein the glucose oxidase has at least 90% amino acid sequence identity to the amino acid sequence of SEQ ID NO: 3.

25. The method of claim 24, wherein the glucose oxidase has the amino acid sequence of SEQ ID NO: 3.
